SEQUENCE LISTING (32 sequences)

SEQ ID NO: 1 - protein, 37 aa, Artificial Sequence
  Synthetic
SMERVEWLRKKLQDVHNFVALGAPLAPRDAGSQRPRK

SEQ ID NO: 2 - protein, 40 aa, Artificial Sequence
  Synthetic
HLNSMERVEWLRKKLQDVHNFVALGAPLAPRDAGSQRPRK

SEQ ID NO: 3 - protein, 47 aa, Artificial Sequence
  Synthetic
LMHNLGKHLNSMERVEWLRKKLQDVHNFVALGAPLAPRDAGSQRPRK

SEQ ID NO: 4 - protein, 48 aa, Artificial Sequence
  Synthetic
QLMHNLGKHLNSMERVEWLRKKLQDVHNFVALGAPLAPRDAGSQRPRK

SEQ ID NO: 5 - protein, 51 aa, Artificial Sequence
  Synthetic
SEIQLMHNLGKHLNSMERVEWLRKKLQDVHNFVALGAPLAPRDAGSQRPRK

SEQ ID NO: 6 - protein, 57 aa, Artificial Sequence
  Synthetic
VKKRSVSEIQLMHNLGKHLNSMERVEWLRKKLQDVHNFVALGAPLAPRDAGSQRPRK

SEQ ID NO: 7 - protein, 44 aa, Artificial Sequence
  Synthetic
SEIQLMHNLGKHLNSMERVEWLRKKLQDVHNFVALGAPLAPRDA

SEQ ID NO: 8 - protein, 41 aa, Artificial Sequence
  Synthetic
QLMHNLGKHLNSMERVEWLRKKLQDVHNFVALGAPLAPRDA

SEQ ID NO: 9 - protein, 40 aa, Artificial Sequence
  Synthetic
LMHNLGKHLNSMERVEWLRKKLQDVHNFVALGAPLAPRDA

SEQ ID NO: 10 - protein, 46 aa, Artificial Sequence
  Synthetic
SEIQLMHNLGKHLNSMERVEWLRKKLQDVHNFVALGAPLAPRDAGS

SEQ ID NO: 11 - protein, 39 aa, Artificial Sequence
  Synthetic
LMHNLGKHLNSMERVEWLRKKLQDVHNFVALGAPLAPRD

SEQ ID NO: 12 - protein, 42 aa, Artificial Sequence
  Synthetic
LMHNLGKHLNSMERVEWLRKKLQDVHNFVALGAPLAPRDAGS

SEQ ID NO: 13 - protein, 41 aa, Artificial Sequence
  Synthetic
KHLNSMERVEWLRKKLQDVHNFVALGAPLAPRDAGSQRPRK

SEQ ID NO: 14 - protein, 43 aa, Artificial Sequence
  Synthetic
LMHNLGKHLNSMERVEWLRKKLQDVHNFVALGAPLAPRDAGSQ

SEQ ID NO: 15 - protein, 44 aa, Artificial Sequence
  Synthetic
NLGKHLNSMERVEWLRKKLQDVHNFVALGAPLAPRDAGSQRPRK

SEQ ID NO: 16 - protein, 49 aa, Artificial Sequence
  Synthetic
KRSVSEIQLMHNLGKHLNSMERVEWLRKKLQDVHNFVALGAPLAPRDAG

SEQ ID NO: 17 - protein, 49 aa, Artificial Sequence
  Synthetic
SVKKRSVSEIQLMHNLGKHLNSMERVEWLRKKLQDVHNFVALGAPLAPR

SEQ ID NO: 18 - protein, 52 aa, Artificial Sequence
  Synthetic
KSVKKRSVSEIQLMHNLGKHLNSMERVEWLRKKLQDVHNFVALGAPLAPRDA

SEQ ID NO: 19 - protein, 52 aa, Artificial Sequence
  Synthetic
FLTKSDGKSVKKRSVSEIQLMHNLGKHLNSMERVEWLRKKLQDVHNFVALGA

SEQ ID NO: 20 - protein, 67 aa, Artificial Sequence
  Synthetic
KVMIVMLAICFLTKSDGKSVKKRSVSEIQLMHNLGKHLNSMERVEWLRKKLQDVHNFVALGAPLAPR

SEQ ID NO: 21 - protein, 79 aa, Artificial Sequence
  Synthetic
AKDMAKVMIVMLAICFLTKSDGKSVKKRSVSEIQLMHNLGKHLNSMERVEWLRKKLQDVHNFVALGAPLAPRDAGSQRP

SEQ ID NO: 22 - protein, 62 aa, Artificial Sequence
  Synthetic
SDGKSVKKRSVSEIQLMHNLGKHLNSMERVEWLRKKLQDVHNFVALGAPLAPRDAGSQRPRK

SEQ ID NO: 23 - protein, 13 aa, Artificial Sequence
  Synthetic
SVSEIQLMHNLGK

SEQ ID NO: 24 - protein, 7 aa, Artificial Sequence
  Synthetic
HLNSMER

SEQ ID NO: 25 - protein, 17 aa, Artificial Sequence
  Synthetic
LDQVHNFVALGAPLAPR

SEQ ID NO: 26 - protein, 8 aa, Artificial Sequence
  Synthetic
ADVNVLTK

SEQ ID NO: 27 - protein, 7 aa, Artificial Sequence
  Synthetic
LMHNLGK

SEQ ID NO: 28 - protein, 11 aa, Artificial Sequence
  Synthetic
FVALGAPLAPR

SEQ ID NO: 29 - protein, 115 aa, Homo sapiens
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 30 - protein, 34 aa, Artificial Sequence
  Synthetic
KHLNSMERVEWLRKKLQDVHNFVALGAPLAPRDA

SEQ ID NO: 31 - protein, 84 aa, Artificial Sequence
  Synthetic
MIPAKDMAKVMIVMLAICFLTKSDGKSVKKRSVSEIQLMHNLGKHLNSMERVEWLRKKLQDVHNFVALGAPLAPRDAGSQRPRK

SEQ ID NO: 32 - protein, 78 aa, Artificial Sequence
  Synthetic
MAKVMIVMLAICFLTKSDGKSVKKRSVSEIQLMHNLGKHLNSMERVEWLRKKLQDVHNFVALGAPLAPRDAGSQRPRK

(19) United States
(12) Patent Application Publication
(10) Pub. No.: US 2013/0217630 A1
(43) Pub. Date: Aug. 22, 2013

(54) PARATHYROID HORMONE VARIANTS AND ASSAYS RELATED TO DISEASE

(71) Applicants: Randall W. Nelson, Phoenix, AZ (US); Paul Oran, Phoenix, AZ (US)

(72) Inventors: Randall W. Nelson, Phoenix, AZ (US); Paul Oran, Phoenix, AZ (US)

(73) Assignee: ARIZONA BOARD OF REGENTS, a body corporate of the State of Arizona, acting for an on behalf of Arizo, Scottsdale, AZ (US)

(21) Appl. No.: 13/820,706

(22) PCT Filed: Oct. 26, 2011
(86) PCT No.: PCT/US11/57862

§ 371 Date: Mar. 29, 2013

Related U.S. Application Data
(60) Provisional application No. 61/406,718, filed on Oct. 26, 2010.

(51) Int. Cl. G01N 33/74

(52) U.S. Cl. CPC G01N 33/74
USPC 514/11.8; 436/501; 530/399; 530/387.9

(57) ABSTRACT

The invention relates to PTH variants and uses thereof in the detection, diagnosis, and treatment of various disease states.

### CROSS REFERNCE

This application claims priority to U.S. Provisional Application Ser. No. 61/406718 filed Oct. 26, 2010, which is incorporated herein by reference in its entirety.

### BACKGROUND

Parathyroid hormone (PTH) is produced in the parathyroid glands through the two-step conversion of pre-pro-PTH (115-amino acids) to pro-PTH (90-amino acids) to the 84-amino acid peptide (PTH1-84). The hormone is secreted into the circulatory system to produce basal (healthy) concentrations of ˜15-65 ng/L (1), and is assayed to assist in the diagnosis of hypo/hyperparathyroidism, hypercalcemia and in monitoring for renal osteodystrophy in patients with end-stage renal failure (1-3). Conventional PTH assays typically rely on two-antibody recognition systems coupled to a variety of detection modalities (e.g., enzymatic amplification, electrochemiluminescence and fluorescence) (4-6). Notably, the most specific assays are able to differentiate between different truncated forms of PTH, and are referred to as “second” or “third” generation PTH assays (7, 8).

Key to the application of these later-generation assays is the ability to selectively monitor different PTH forms of known biological consequence. In particular, two variants, full-length PTH1-84 and PTH missing the six N-terminal amino acids (PTH7-84), are the subject of increased clinical investigation and potential diagnostic capability. Due to the inability of existing assays to detect microheterogeneity, these variants were historically considered as a single PTH value (i.e., the “first” generation assays) (7,9). The classification of each as its own molecular entity with the ability to analyze and study each independently, suggests an antagonistic relationship between the two different forms in relationship to calcium homeostasis (10). In fact, there is mounting clinical evidence that the ratio between PTH1-84 and PTH7-84 may differentiate between hyperparathyroid bone turnover and adynamic bone disease (11-13).

The PTH1-84-to-PTH7-84 paradigm is a recent example of describing the biological and clinical utility of microheterogeneity within the PTH protein. PTH1-34, has been identified as an in vivo variant that exhibits biochemical activity comparable to the full-length protein. Consequently, it represents a classic example of a peptide-based bioactive variant that has transitioned through drug development to the point of FDA-approval for the treatment of osteoporosis (rPTH1-34 (teriparatide)) (14-16). Collectively the monitoring of these clinically-relevant PTH variants—PTH1-84, PTH7-84 and PTH1-34—is achieved through three separate, highly-specific immunometric assays. However, even greater microheterogeneity may exist within PTH, which has yet to be fully characterized to determine clinical utility and/or confounding effects on present-day assays. The accurate examination of known PTH variants, while simultaneously evaluating other possible variants, requires a degree of analytical freedom that universally escapes conventional assays.

Current assays do not capture the full information content of a target analyte (and its variants), which often results in ambiguities in the exact molecular species under investigation. Because upstream causes and downstream effects of disease are often due to such structural modifications, these limitations represent significant problems during the analysis of clinically significant analytes

### SUMMARY OF THE INVENTION

The invention relates to PTH variants and uses thereof in the diagnosis and treatment of various disease states.

In a first aspect, the invention relates to methods of detecting a PTH-related disease in a patient, comprising detecting the presence of one or more PTH variants in a biological sample from a patient, wherein the one or more PTH variants is selected from the group consisting of PTH 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22), and wherein the presence of one or more variants in the biological sample is indicative of a PTH-related disease in the patient.

In a second aspect, the invention relates to isolated polypeptides selected from the group consisting of PTH 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22).

In a third aspect, the invention relates to an isolated binding molecule which selectively binds to a single PTH variant selected from the group consisting of PTH 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22).

In a fourth aspect, the invention relates to pharmaceutical compositions comprising
(a) PTH variants 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22); and
(b) a pharmaceutically acceptable carrier.

In a fifth aspect, the invention relates to methods for treating a PTH-related disease, comprising administering to a patient in need thereof, a therapeutically effective amount to treat the PTH related disease, of one or more polypeptides selected from the group consisting of PTH variants 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22)

In a sixth aspect the invention relates to methods for identifying polypeptide variants in a biological sample comprising:
(a) capturing the polypeptide variants from the sample using affinity capture; and
(b) identifying the polypeptide variants using a polypeptide separation assay.

### BRIEF DESCRIPTION OF THE DRAWINGS

FIG. 1. PTH Variant Map. Top panel: A. N-terminally truncated PTH variants identified previously [e.g. Ref 11 and 24]. B. Variants added to map during this study using full-scan MSIA (MALDI-TOFMS) . With the exception of PTH7-84, all variants depicted in (A) and (B) were observed in the majority of clinical samples under investigation. Bottom panel: MSIA (MALDI-TOFMS) spectra representative of the 12 renal failure samples (blue) and the 12 healthy controls (red). The following species were consistently found at higher frequency relative abundance in the renal failure cohort (m/z observed; calculated): PTH1-84 (9426.03; 9425.71), PTH28-84 (6179.61; 6179.91), PTH34-84 (5472.91; 5473.16), PTH37-84 (5155.71; 5155.77), PTH38-84 (5051.92; 5051.61), PTH34-77 (4715.98; 4716.26), PTH37-77 (4397.71; 4398.87), PTH45-84 (4379.65; 4379.82), PTH38-77 (4285.57; 4285.71) and PTH48-84 (4136.42; 4136.60).

FIG. 2 Use of the quantitative data to create ROC curves (receiver operating characteristic curve) for the three PTH variants.

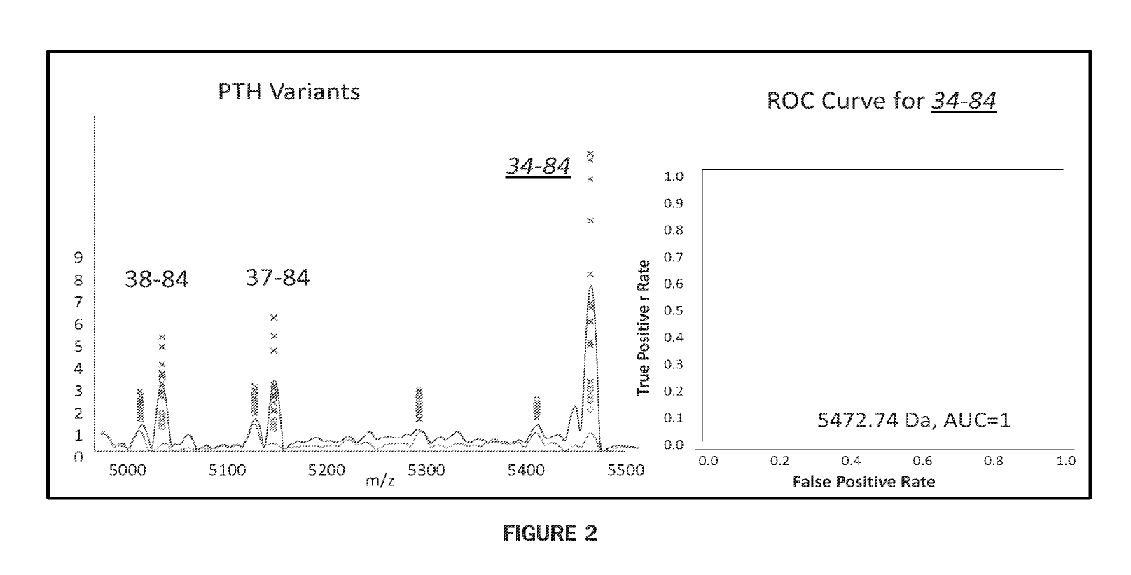

FIG. 3. PTH MSIA spectrum from an individual with hypercalcemia and renal disease. PTH variants were detected (m/z observed; calculated): PTH (44-77) (3778.8, 3778.1), PTH (38-76) (4186.3, 4185.6), PTH (38-77) (4285.4, 4284.7), PTH (45-84) (4379.5, 4378.8), PTH (44-84) (4535.4, 4535.0), PTH (34-77) (4715.8, 4715.2), PTH (41-84) (4817.6, 4816.4), PTH (34-79) (4929.5, 4929.8), PTH (38-84) (5042.9,5041.6), PTH (37-84) (5156.0, 5154.8), and PTH (34-84) (5473.2, 5472.1).

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the invention relates to methods of detecting a PTH-related disease in a patient, comprising detecting the presence of one or more PTH variants in a biological sample from a patient, wherein the one or more PTH variants is selected from the group consisting of PTH 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22), and wherein the presence of the one or more variants in the biological sample is indicative of a PTH-related disease in the patient.

The methods of this aspect of the invention can be used, for example, to measure an individual's specific PTH variant phenotype for the purpose of predicting, diagnosing, or monitoring a PTH-related disease and which can be further used to determine the relative abundance of the PTH variant phenotype as they relate to a PTH-related disease. As used herein, “is indicative of a PTH-related disease” means that the PTH variants detected provide information to aid an attending practitioner in determining (a) whether a patient has a PTH-related disease, (b) whether a patient is predisposed to developing a PTH-related disease, (c) progression of the PTH-related disease in the patient, and/or (d) determining patient response to therapeutic treatment for a PTH-related disease.

As used herein a “PTH-related disease” is any disease resulting from dysfunction of the parathyroid glad and alterations in parathyroid hormone. These include, but are not limited to primary hyperparathyroidism, secondary hyperparathyroidism, hypoparathyroidism, hypocalcemia, hypercalcemia, adynamic bone disease, vitamin D deficiency, renal osteodystrophy (chronic renal failure), diabetes, kidney disease, bone disease, osteoporosis, and parathyroid cancer.

In various embodiments, the detection of 1, 2, 3, 4, 5, 6, 7, 8, 9, 10, 11, 12, 13, 14, 15, 16, 17, 18, or all of the 19 PTH variants recited is indicative of a PTH-related disease.

Any suitable method of detecting the presence of the one or more PTH variants can be used. Detection of the PTH variants can be sequential or simultaneous, preferably simultaneous. In one embodiment, the individual PTH variants can be uniquely and individually identified using probes specific for each of the individual PTH variants. In this embodiment, and based on the teaching herein, the novel isolated PTH variant polypeptides can be used to generate PTH variant-specific binding agents, including but not limited to aptamers, antibodies, small molecules, or the like. Once developed, the PTH variant-specific binding agents could be used as probes which could then be used in combination with other suitable detection methods well known to the skilled artisan. These include, but not limited to, immunohistochemical staining of (tissue) samples, flow cytometric detection, scanning laser cytometric detection, fluorescent immunoassays, enzyme-linked immunosorbent assays (ELISAs), radioimmunoassays (RIAs), bioassays (e.g., neutralization assays), Western blotting applications, microarrays, affinity chromatography, surrogate peptide detection assays (SISCAPA), etc. In this embodiment, the detection of 1, 2, 3, 4, 5, 6, 7, 8, 9, 10, 11, 12, 13, 14, 15, 16, 17, 18, or all of the 19 PTH variants, using PTH variant-specific binding agents, is indicative of a PTH-related disease.

In another embodiment, detection of the PTH variants can be accomplished by affinity capture of two or more of the PTH variants from the sample, such as by use of a PTH antibody, followed by the separation and identification of the individual PTH variants. In various embodiments, the detection of 1, 2, 3, 4, 5, 6, 7, 8, 9, 10, 11, 12, 13, 14, 15, 16, 17, 18, or all 19 PTH variants using PTH variant capture and identification is indicative of a PTH-related disease.

The non-specific capture of two or more of the PTH variants can be accomplished using any suitable method and are well known to the skilled artisan, including, but not limited to, use of PTH binding molecules including but not limited to antibodies, receptors, proteins, small molecules, aptamers or the like. In a preferred embodiment, affinity capture comprises immunoassay embodiments of the invention involve the use of general antibodies for PTH, which bind to 2 or more the PTH variants. Any suitable PTH antibody may be used, such as those known in the art. As demonstrated in the following Examples, common polyclonal antibodies may be used for capturing PTH and PTH variants from a patient sample. Alternatively, monoclonal antibodies specific for PTH also may be used in non-specific PTH affinity capture as long as the antibody recognizes two or more of the recited PTH variants.

Once captured, the PTH variants are then separated and individually identified using a polypeptide separation technique. any suitable polypeptide separation technique can be used, including but not limited to, chromatographic assays, ultrafiltration assays, two-dimensional gel electrophoresis, ion mobility separation, capillary isoelectric focusing, mass spectrometry, matrix-assisted laser desorption/ionization (MALDI) time-of-flight mass spectrometry (TOF), Electronspray ionization (ESI) time of flight spectrometry (TOF), Liquid Chromatography-Mass Spectrometry (LC/MS), or combinations thereof

In a preferred embodiment, the PTH variants are captured using PTH affinity capture using PTH specific antibodies (ie: not selective for any of the variants) and the PTH variants are identified using mass spectrometry. In a further, preferred embodiment, the mass spectrometry method is Matrix-assisted laser desorption/ionization (MALDI) using a time-of-flight mass spectrometer or MADLI-TOF MS. In various embodiments, the detection of 1, 2, 3, 4, 5, 6, 7, 8, 9, 10, 11, 12, 13, 14, 15, 16, 17, 18, or all 19 PTH variants using affinity capture coupled with mass spectrometry is indicative of a PTH-related disease.

Suitable biological samples for use in the methods of the invention include serum, plasma, cerebrospinal fluid, pleural fluid, joint fluid, nipple discharge, saliva. In a preferred embodiment, the patient sample is serum or plasma.

In another embodiment, the methods comprise quantifying an amount of the one or more recited PTH variants, wherein the quantification can be used to determine the progression or prognosis of the PTH-related disease. Thus, in methods for detecting two or more PTH variants, at least two PTH-disease-related metrics can be obtained—1) the PTH-variant phenotype indicative of the presence of the disease and 2) the abundance of the PTH-variants indicative of the progression or prognosis of the PTH-related disease. The data so obtained can thus be used for the prediction and diagnosis of PTH-related disease, as well as in the subsequent monitoring of PTH-related disease progression and the effect of therapy for a PTH-related disease in a patient. Thus, in one embodiment, the presence of any amount of antibodies to one or more of the recited PTH variants, or the one or more PTH variants themselves in a sample from a patient can indicate a PTH-related disease in the patient. In another embodiment, if antibodies to one or more of the recited PTH variants, or the one or more PTH variants themselves are present in a sample from a patient at risk of PTH-related disease, at levels which are higher than that of a control sample (i.e. a sample from a patient who does not have a PTH-related disease) than the patient at risk of PTH-related disease has a likelihood of a PTH-related disease. If appropriate, the presence of the PTH-related disease can then be confirmed using diagnostic techniques known to the skilled artisan.

An additional embodiment of the methods of the invention is the use of quantitative data from the PTH variants to monitor disease progression (or effective treatment). One way to use the quantitative data is to create ROC curves (receiver operating characteristic curve) for the one or more individual variants. The methods of the present invention may be used in the diagnosis and prognostic determination of any PTH-related disease in which PTH levels are altered. In one embodiment, a reduction in the quantity of one or more variants is provides information on treatment efficacy.

In a preferred embodiment the one or more captured PTH variants, as well as the full length PTH polypeptide, are subjected to identification and characterization, including quantification, using mass spectrometry. Data resulting from the capture and identification indicate a PTH variant phenotype which is indicative of the presence of the PTH-related disease. The measurement also yields the quantity of the PTH variants, which is indicative of the progress of the PTH-related disease. Suitable methods for the separation, detection, and quantification of PTH variants are discussed herein, preferably comprising mass spectrometry. In this embodiment, the detection and quantification of 1, 2, 3, 4, 5, 6, 7, 8, 9, 10, 11, 12, 13, 14, 15, 16, 17, 18 or all 19 PTH variants is indicative of a PTH-related disease.

In a further embodiment the presence of the full length PTH polypeptide (SEQ ID NO: 29) and/ or one or more of PTH variants 45-84 (SEQ ID NO: 2), 38-84 (SEQ ID NO: 3), 37-84 (SEQ ID NO: 4), 34-84 (SEQ ID NO: 5), 1-84 (SEQ ID NO: 31), and 7-84 (SEQ ID NO: 32) are also detected. These variants have also been associated with PTH-related diseases.

In another embodiment, the method further comprises determining a ratio of individual PTH variants to each other and/or to the full length PTH polypeptide. The detection and monitoring of these ratios could then be used in the diagnosis and prognostic and treatment efficacy determinations of PTH-related disease. In this embodiment, the detection, quantification, and ratios of 2, 3, 4, 5, 6, 7, 8, 9, 10, 11, 12, 13, 14, 15, 16, 17, 18 or all 19 PTH variants and/or the full length PTH polypeptide is indicative of a PTH-related disease diagnosis, progression and treatment efficacy.

The variants that can be detected, identified, and quantified using the PTH detection methods of the invention as well as the populational frequencies for detectable, identifiable, and quantifiable variants (which addresses the question of the number of samples (of the total) in which a particular protein variant was observed), is outlined in Table 1.

TABLE 1
PTH Variant Frequencies (MALDI-TOF-MS)

| | | | | | | | | | | | | | |
|---|---|---|---|---|---|---|---|---|---|---|---|---|---|
| Variant | [M] | 1 | 2 | 3 | 4 | 5 | 6 | 7 | 8 | 9 | 10 | 11 | 12 |

Renal Failure Samples

| | | | | | | | | | | | | | |
|---|---|---|---|---|---|---|---|---|---|---|---|---|---|
| 48-84 | 4135.596 | x | x | x | x | x | x | x | x | x | x | x | x |
| 45-84 | 4378.815 | | x | x | x | | x | x | | | x | x | x |
| 38-84 | 5041.603 | x | x | x | x | x | x | x | x | x | x | x | x |
| 37-84 | 5154.762 | x | x | x | x | x | x | x | x | x | x | x | x |
| 34-84 | 5472.149 | x | x | x | x | x | x | x | x | x | x | x | x |
| 28-84 | 6178.904 | | x | x | x | x | x | x | x | x | x | x | x |
| iPTH | 9424.716 | x | x | x | x | x | x | x | x | x | x | x | x |
| 34-77 | 4715.25 | | x | x | x | x | | x | x | | x | x | x |
| 37-77 | 4397.863 | | x | x | x | | | | x | x | x | x | x |
| 38-77 | 4284.704 | | x | x | x | x | x | | x | x | x | x | x |
| 34-79 | 4929.514 | | x | x | x | x | x | x | x | x | x | x | x |
| 38-76 | 4185.6 | | x | | x | | | | x | | x | x | x |
| 38-79 | 4499 | | | | | | | | | x | | x | x |
| 44-84 | 4535 | | x | | | | | | | | | x | x |
| 38-80 | 4627.1 | | | | | x | | x | | x | | | |
| 41-84 | 4816.4 | x | x | x | | x | x | x | x | x | x | | x |
| 30-78 | 5293.9 | x | x | | | | | | | | | | x |
| 27-75 | 5336.9 | | | | | | | | x | | | | |
| 26-77 | 5678.4 | | x | | | | | | x | | | x | x |
| 19-70 | 5905.7 | | x | | | x | x | | x | | x | x | x |
| 9-75 | 7566.5 | x | | x | | x | | | | | | | |
| 4-82 | 8936.2 | | | x | | x | | x | | | | | |
| 23-84 | 6890.8 | x | | x | x | | x | | x | | | | x |

Normal Samples

| | | | | | | | | | | | | |
|---|---|---|---|---|---|---|---|---|---|---|---|---|
| 48-84 | 4135.596 | x | | x | x | x | x | x | x | x | x | x |
| 45-84 | 4378.815 | | | x | | | | | | | | |
| 38-84 | 5041.603 | | | | | x | | | | | | |
| 37-84 | 5154.762 | | | | | | | | | | | |
| 34-84 | 5472.149 | x | x | | | x | | x | x | | | x |
| 28-84 | 6178.904 | | | | | | | | | | | |
| iPTH | 9424.716 | | | | | | | | | | x | x |
| 34-77 | 4715.25 | | | | | | | | | | | |
| 37-77 | 4397.863 | x | | x | | x | x | | x | | | |
| 38-77 | 4284.704 | x | | | | | | | x | | | |
| 34-79 | 4929.514 | | | | | | | | x | | | |
| 38-76 | 4185.6 | x | | | | | | | | | | |
| 38-79 | 4499 | | | | | | | | | | | |
| 38-80 | 4627.1 | | | | | | | | | | | |
| 41-84 | 4816.4 | | | | | | | | | | | |
| 30-78 | 5293.9 | | | | | | | | | | | |
| 26-77 | 5678.4 | | | | | | | | | | | |
| 19-70 | 5905.7 | | | | | | | | | | | x |
| 9-75 | 7566.5 | | | | | | | | | | | |
| 4-82 | 8936.2 | | | | | | | | | | | |
| 23-84 | 6890.8 | | | | | | | | | | | |

The preferred embodiments outlined below are based upon the results from Table 1 showing which variants were present in each of the individual samples from renal failure patients.

In a preferred embodiment, the detection of one or more PTH variants selected from the group consisting of PTH (48-84)(SEQ ID NO: 1), (28-84)(SEQ ID NO: 6), (34-77)(SEQ ID NO: 7), (37-77)(SEQ ID NO: 8), (38-77)(SEQ ID NO: 9), (34-79) (SEQ ID NO: 10), (44-77) (SEQ ID NO: 30) and (41-84) (SEQ ID NO: 15) is indicative of a PTH-related disease. In this embodiment, the detection of 1, 2, 3, 4, 5, 6, 7, or all 8 PTH variants is indicative of a PTH-related disease.

In a further preferred embodiment, the presence of one or more PTH variants selected from the group consisting of 48-84 (SEQ ID NO: 1), 45-84 (SEQ ID NO: 2), 38-84 (SEQ ID NO: 3), 37-84 (SEQ ID NO: 4), 34-84 (SEQ ID NO: 5), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30)and 23-84 (SEQ ID NO: 22) is indicative of renal osteodystrophy, as demonstrated in the Examples that follow and the teachings herein. In this embodiment, the detection of 1, 2, 3, 4, 5, 6, 7, 8, 9, 10, 11, 12, 13, 14, 15, 16, 17, 18, 19, 20, 21, 22, or all 23 PTH variants is indicative of a renal osteodystrophy.

In a another preferred embodiment, presence of one or more PTH variants selected from the group consisting of 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30)and 23-84 (SEQ ID NO: 22) is indicative of renal osteodystrophy, as demonstrated in the Examples that follow and the teachings herein. In this embodiment, the detection of 1, 2, 3, 4, 5, 6, 7, 8, 9, 10, 11, 12, 13, 14, 15, 16, 17, 18, or all 19 PTH variants is indicative of a renal osteodystrophy.

In a further preferred embodiment, the presence of PTH variant 44-77 (SEQ ID NO: 30) is indicative of hypercalcemia, as demonstrated in the Examples that follow.

The definitions and embodiments disclosed in all other aspects of the invention, apply to this aspect as well.

In a second aspect, the invention relates to an isolated polypeptide selected from the group consisting of PTH 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22).

The polypeptides of the invention are novel PTH variants identified by the inventors, which can be used, for example, to develop PTH variant-specific binding agents and/or reagents for use in the diagnostic and prognostic methods of the invention. The isolated polypeptides can also be used as standards in the diagnostic methods of the invention. The isolated polypeptides may also be used, for example, as therapeutics for treating disorders such as PTH-related diseases including, but not limited to primary hyperparathyroidism, secondary hyperparathyroidism, hypoparathyroidism, hypocalcemia, hypercalcemia, adynamic bone disease, vitamin D deficiency, renal osteodystrophy (chronic renal failure), diabetes, kidney disease, bone disease, osteoporosis, and parathyroid cancer.

As used herein, “isolated” means that the polypeptides are removed from their normal surrounding sequences in the protein, and are substantially free of contaminating material used to isolate them. The isolated polypeptides may be stored in any suitable state, including but not limited to in solution. The isolated polypeptides may be chemically synthesized using means known in the art, or may be prepared by standard recombinant expression methods, based on the teachings herein.

The term “polypeptide” is used in its broadest sense to refer to a polymer of subunit amino acids, amino acid analogs, or peptidomimetics, including proteins and peptoids. The polypeptides may be naturally occurring full length proteins or fragments thereof, processed forms of naturally occurring polypeptides (such as by enzymatic digestion), chemically synthesized polypeptides, or recombinantly expressed polypeptides. The polypeptides may comprise D- and/or L-amino acids, as well as any other synthetic amino acid subunit, and may contain any other type of suitable modification, including but not limited to peptidomimetic bonds and reduced peptide bonds.

In certain embodiments, the polypeptides of the invention may further comprise a tag, such as a detectable moiety. The tag(s) can be linked to the polypeptide through covalent bonding, including, but not limited to, disulfide bonding, hydrogen bonding, electrostatic bonding, recombinant fusion and conformational bonding. Alternatively, the tag(s) can be linked to the polypeptide by means of one or more linking compounds. Techniques for conjugating tags to polypeptides are well known to the skilled artisan. However, they may also be used for other detection and/or analytical purposes. Any suitable detection tag can be used, including but not limited to enzymes, prosthetic groups, fluorescent materials, luminescent materials, bioluminescent materials, radioactive materials, positron emitting metals, and nonradioactive paramagnetic metal ions.

The definitions and embodiments disclosed in all other aspects of the invention, apply to this aspect as well.

In a third aspect, the invention relates to isolated binding molecules which selectively bind to a single PTH variants selected from the group consisting of PTH 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22).

An isolated binding molecule of this aspect of the invention is specific for a single PTH variant and can thus be used as a probe, as described above, in the diagnostic and prognostic methods of the invention.

In a preferred embodiment, an isolated binding molecule of this aspect of the invention selectively binds a single isolated polypeptide of the second aspect of the invention.

In various embodiments, the binding molecules, include, but are not limited to aptamers, antibodies, small molecules, or the like. In a preferred embodiment the isolated binding molecule is a PTH varaint-specific antibody.

The definitions and embodiments disclosed in all other aspects of the invention, apply to this aspect as well.

In a fourth aspect, the invention relates to pharmaceutical compositions comprising:
(a) one or more substantially purified polypeptide selected from the group consisting of PTH variants 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22); and
(b) a pharmaceutically acceptable carrier.

For administration, the polypeptides are ordinarily combined with one or more adjuvants appropriate for the indicated route of administration. The polypeptides may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, dextran sulfate, heparin-containing gels, and/or polyvinyl alcohol, and tableted or encapsulated for conventional administration. Alternatively, the polypeptides may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The polypeptides may be made up in a solid form (including granules, powders or suppositories) or in a liquid form (e.g., solutions, suspensions, or emulsions). The polypeptides may be applied in a variety of solutions. Suitable solutions for use in accordance with the invention are sterile, dissolve sufficient amounts of the polypeptides, and are not harmful for the proposed application.

Alternatively, the pharmaceutical composition may comprise in addition to the polypeptide of the invention (a) a lyoprotectant; (b) a surfactant; (c) a bulking agent; (d) a tonicity adjusting agent; (e) a stabilizer; (f) a preservative and/or (g) a buffer.

In various embodiments the pharmaceutical compositions are used to treat PTH-related diseases, including, but not limited to primary hyperparathyroidism, secondary hyperparathyroidism, hypoparathyroidism, hypocalcemia, hypercalcemia, adynamic bone disease, vitamin D deficiency, renal osteodystrophy (chronic renal failure), diabetes, kidney disease, bone disease, osteoporosis, and parathyroid cancer.

In various embodiments, 1, 2, 3, 4, 5, 6, 7, 8, 9, 10, 11, 12, 13, 14, 15, 16, 17, 18 or all 19 of the isolated polypeptides may be used in the pharmaceutical compositions. The pharmaceutical compositions may also include the full length PTH polypeptide (SEQ ID NO: 29) and/or one or more of the PTH variants 45-84 (SEQ ID NO: 2), 38-84 (SEQ ID NO: 3), 37-84 (SEQ ID NO: 4), 34-84 (SEQ ID NO: 5), 1-84 (SEQ ID NO: 31), and 7-84 (SEQ ID NO: 32).

The definitions and embodiments disclosed in all other aspects of the invention, apply to this aspect as well.

In a fifth aspect, the invention relates to methods for treating a PTH-related disease, comprising administering to a patient in need thereof, a therapeutically effective amount to treat the PTH-related disease of one or more polypeptides selected from the group consisting of PTH variants 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22).

As used herein, a “therapeutically effective amount” refers to an amount of the polypeptide that is effective for treating and/or limiting a PTH -related disease, including, but not limited to primary hyperparathyroidism, secondary hyperparathyroidism, hypoparathyroidism, hypo/hypercalcemia, adynamic bone disease, vitamin D deficiency, renal osteodystrophy (chronic renal failure), diabetes, kidney disease, bone disease, osteoporosis, and cancer (e.g. parathyroid cancer).

The polypeptides are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Dosage regimens can be adjusted to provide the optimum desired response (e.g., a therapeutic or prophylactic response). A suitable dosage range may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. The polypeptides can be delivered in a single bolus, or may be administered more than once (e.g., 2, 3, 4, 5, or more times) as determined by an attending physician.

In various embodiments, the methods comprise administering 1, 2, 3, 4, 5, 6, 7, 8, 9, 10, 11, 12, 13, 14, 15, 16, 17, 18 or all 19 of the PTH polypeptide variants. The method may also comprise administering the full length PTH polypeptide (SEQ ID NO: 29) and/or one or more of the PTH polypeptide variants 45-84 (SEQ ID NO: 2), 38-84 (SEQ ID NO: 3), 37-84 (SEQ ID NO: 4), 34-84 (SEQ ID NO: 5), 1-84 (SEQ ID NO: 31), and 7-84 (SEQ ID NO: 32).

In a further preferred embodiment, the methods comprise administering one or more of the PTH polypeptide variants selected from the group consisting of PTH 48-84 (SEQ ID NO: 1), 28-84 (SEQ ID NO: 6), 44-84 (SEQ ID NO: 13), 41-84 (SEQ ID NO: 15), and 23-84 (SEQ ID NO: 22).

The definitions and embodiments disclosed in all other aspects of the invention, apply to this aspect as well.

In a sixth aspect the invention relates to methods for identifying polypeptide variants in a biological sample comprising:
(a) capturing the polypeptide variants from the biological sample using affinity capture; and
(b) identifying the polypeptide variants using a polypeptide separation assay.

Any one gene can produce multiple, qualitatively different proteins in varying amounts when posttranslational modifications are considered. Thus, an individual's protein phenotype (for any one gene-gene product combination) contains additional qualitative, quantitative and temporal components. The protein phenotype builds upon the initial description of an in vivo protein by considering, e.g., quantitative modulations in the posttranslational variants. The ideal single-measurement assay is able to monitor several molecular variants of products from a given gene, revealing an individuals' protein phenotype relative to disease

The polypeptide variants identified using the methods of the invention can be used individually and/or combinatorially in the detection, diagnosis, progression monitoring and treatment of many disorders or diseases. The methods can be used to measure an individual's specific protein phenotype and the relative abundance of the protein phenotype(s) as they relate to a disease. This method allows for the identification of posttranslational variants for any clinical relevant polypeptides which may be associated with a disease state.

As used herein the term “variant” can be used to describe any truncated form of the polypeptide, including both N-terminal and C-terminal truncations and can range from 2 amino acid residue polypeptides to the full length polypeptide.

Affinity capture uses an antibody or antibodies to the full length or fragmented polypeptide(s) of interest, in order to capture all of the variants from the sample. The polypeptide variants are then separated and identified using a separation assay as outlined in the first aspect of the invention.

In a preferred embodiment, the method is used to identify PTH variants or to identify the heterogeneity of PTH variants in a biological sample.

In a further embodiment the method comprises:
(a) capturing the PTH variants using PTH affinity capture; and
(b) identifying one or more PTH variants using mass spectroscopy.

As used herein the term “heterogeneity” means diversity of different molecular species that are very similar in structure.

The definitions and embodiments disclosed in all other aspects of the invention, apply to this aspect as well.

In one embodiment the full length PTH polypeptide or the PTH variants can be digested using enzymatic digest and the resulting enzymatic product variants can be monitored. Enzymatic digest can be accomplished using any suitable digestive enzyme, including but not limited to, Trypsin. Thus, the PTH variants can further include enzymatic digest products that result from an enzymatic digest, starting with full length PTH variants. These include but are not limited to SVSEIQLMHNLGK (aa1-13) (SEQ ID NO: 23), HLNSMER (aa14-20) (SEQ ID NO: 24), LDQVHNFVALGAPLAPR (aa28-44) (SEQ ID NO: 25), ADVNVLTK (aa73-80) (SEQ ID NO: 26), LMHNLGK (aa7-13) (SEQ ID NO: 27) and FVALGAPLAPR (aa34-44) (SEQ ID NO: 28).

### EXAMPLES

Described here is the invention of a high throughput, multiplexed assay that in a single analysis detects PTH1-84, C-PTH variants (e.g. PTH34-84), and C-terminally truncated variants (e.g. 38-77) using, for example, mass spectrometry (at least 23+ variants total). The assay was successful in stratifying renal failure samples from matched healthy controls (with ROCAUC >0.80 for the majority of variants described). The variants described here, used individually and/or combinatorially, have clinical utility in the diagnosis several diseases including: primary hyperparathyroidism, secondary hyperparathyroidism, hypoparathyroidism, hypocalcemia, hypercalcemia, adynamic bone disease, vitamin D deficiency, renal osteodystrophy (chronic renal failure), diabetes, and cancer (e.g. parathyroid cancer).

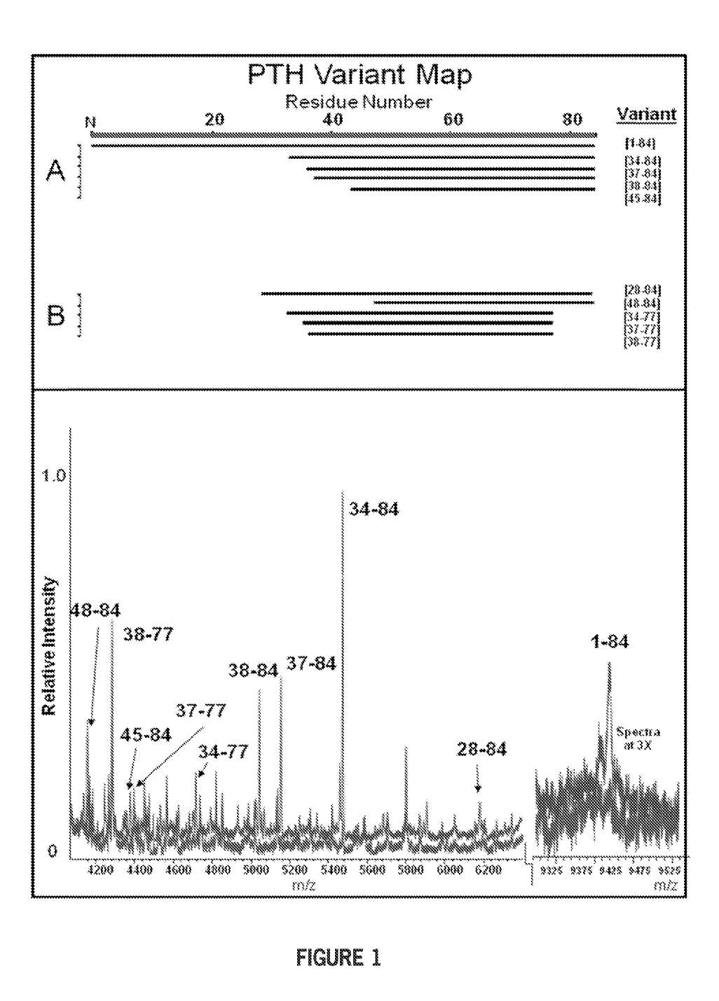

These examples focus on the simultaneous analysis of PTH variants—collectively referred to as Parathyroid Hormone (Swissprot accession# P01270), for the diagnosis of renal impairment and end-stage renal disease (collectively labeled as renal failure). The two cohorts under investigation were healthy (not known to have ailments; n=12) and renal failure (n=12). During the course of investigation, PTH protein variants were analyzed from blood plasma using immunoaffinity extraction followed by mass spectrometry. FIG. 1 shows an example PTH variant map that includes many of the PTH variants discovered (see Table 1 for the complete list of PTH variants that were identified). Top panel: A. list of all the N-terminally truncated PTH variants identified previously by others that were detected in this study B. Some of the variants discovered and added to the map during this study using full-scan MSIA (MALDI-TOF-MS). All variants depicted in (A) and (B) were observed in the majority of clinical samples under investigation. Bottom panel: MSIA (MALDI/TOFMS) spectra representative of the 12 renal failure samples (blue) and the 12 healthy controls (red). The following species, as seen labeled in the figure, were consistently found at higher frequency relative abundance in the renal failure cohort (m/z observed; calculated): PTH1-84 (9426.03; 9425.71), PTH28-84 (6179.61; 6179.91), PTH34-84 (5472.91; 5473.16), PTH37-84 (5155.71; 5155.77), PTH38-84 (5051.92; 5051.61), PTH34-77 (4715.98; 4716.26), PTH37-77 (4397.71; 4398.87), PTH45-84 (4379.65; 4379.82), PTH38-77 (4285.57; 4285.71) and PTH48-84 (4136.42; 4136.60).

### Example 1

### Materials and Methods

Approach: As a starting point for the development of the PTH MSIA, we surveyed the literature to define molecular variants that have already been identified. In addition to the well-characterized truncated variants (e.g., PTH1-84 and 7-84), four other molecular versions have been reported in the literature as present in human biofluids (primarily plasma or serum). Aligning these fragments to the sequence of PTH1-84 produced a variant map revealing forms stemming predominantly from N-terminal truncations (FIG. 1A). A conserved region (among several variants) was evident between residues 48-84. This region was suitable for immunoaffinity targeting in order to capture ragged N-terminal variants (e.g., PTH1-84 and PTH7-84). A full-scan MSIA (MALDI-TOF-MS) (18-23) captured these variants, and also presented the opportunity to discover other variants immunoreactive with the antibody (FIG. 1B). Reagents: Goat polyclonal Anti PTH39-84 antibody was purchased from Immutopics International (San Clemente, Calif.). Recombinant human PTH (rhPTH) was obtained from Bachem (Torrance, Calif.). Premixed MES-buffered saline powder packets were from Pierce (Rockford, Ill.). Extraction of PTH from plasma was carried out with proprietary MSIA pipette tips (MSIA-Tips) from Intrinsic Bioprobes (Tempe, Ariz.) derivatized with the PTH antibodies via 1,1′ Carbonyldiimidazole (CDI) chemistry as described below. Premade 10×0.1M Hepes-buffered saline buffer (HBS-N) with 30 mM EDTA and 0.2% (v/v) surfactant P20 (HBS-EP) were purchased from BIACORE (Piscataway, N.J.). Synthetic heavy labeled peptides were obtained from Thermofisher (Ulm, Germany). All other chemicals were obtained from Sigma-Aldrich (St. Louis, Mo.).

Samples: Twenty four serum samples representing twelve individuals diagnosed with either sever renal impairment or end-stage renal disease (10 males and 2 females; mean age 66.7) and twelve healthy individuals (10 males and 2 females; mean age 65) were used in the study. The renal failure samples represented 3 Hispanics, 2 Asians, 2 African Americans, and 6 Caucasians. The ethnicity information for the healthy samples was not available. Sample Preparation and Immunocapture: MSIA-Tips were prepared by Intrinsic Bioprobes as previously described (18-23). Prior to analysis samples were thawed to 25 ° C. using a water bath and one mL of serum (or plasma) was diluted with 750 μL HBS-EP buffer to result in a total analytical volume of 1.75 mL. PTH was then extracted with the aid of a Beckman Multimek 96 pipetting robot by repeatedly (1,500 repetitions) drawing and expelling (back into the analytical volume) 125 μL aliquots of the analytical volume through the antibody prelinked MSIA-Tip. After extraction, the pipettes were rinsed using HBS-EP and H₂O, (in this order, each rinse=15 repetitions of 150 μL), after which PTH was either immediately eluted for detection with MALDI-TOF-MS or digested and analyzed using High Resolution LC-MS/MS or Selected Reaction Monitoring as described below.

MALDI-TOF Mass Spectrometry: Samples were prepared for MALDI-TOF MS by drawing 4 μL of MALDI matrix solution (saturated aqueous solution of sinapic acid, in 33% (v/v) acetonitrile, 0.4% (v/v) trifluoroacetic acid, TFA) into the pipette and depositing onto a MALDI-TOF-MS target (17). MALDI-TOF-MS was performed using a Bruker Ultraflex MALDI-TOF instrument operating in the positive ion, delayed-extraction mode; reflector engaged with ‘ion source 1’ at 25.00 kV, ‘ion source 2’ at 21.90 kV, lens at 9.50 kV, ‘reflector’ at 26.30 kV, ‘reflector 2’ at 13.80, 340 ns delayed extraction, deflection signal suppression up to m/z 4000, and 2 GS/s sample rate. Ten thousand laser-shots were signal averaged for each mass spectrum using a laser repetition rate of 100 Hz. Spectra were externally calibrated with a mixture of 4 proteins supplied by Bruker (Cat. No. 208241) ranging in average m/z from 5734.52 (Insulin [M+H]⁺) to 12,360.97 (Cytochrome C [M+H]⁺).

### Example 2

### Results

Top-Down Analysis and Discovery of Novel Variants. The approach described herein couples targeting a common region of PTH using a polyclonal antibody (raised to the C-terminal end of the protein) with subsequent identification using mass spectrometry (either MALDI-TOF-MS or SRM). Using a top-down approach (MALDI-TOF-MS), novel truncated PTH variants were discovered in clinical samples (FIG. 1). FIG. 1, Bottom panel shows two MSIA (MALDI-TOF-MS) spectra representative of those obtained from plasma samples of 12 individuals suffering from renal failure and 12 healthy controls. Signals corresponding to the previously reported N-terminally truncated variants as well as additional signals aligning with other novel variants are indicated. Notably, a conserved cleavage site (residue 77) was observed in several of these new variants (FIG. 1B). With the exception of PTH7-84, the variants depicted in FIGS. 1A and B were detected in the majority of clinical samples, and not readily evident in the control samples (Table 1).

### Example 3

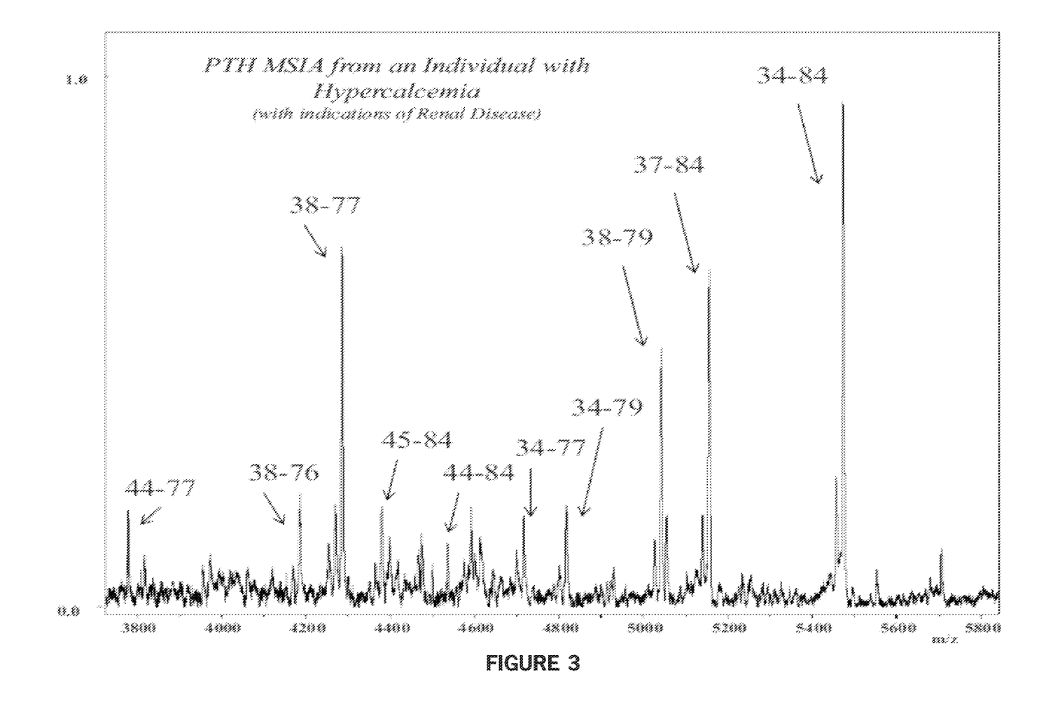

FIG. 3 presents a MALDI-TOF-MS spectrum from an individual known to have hypercalcemia (with indications of renal disease). The results confirm the PTH heterogeneity signature seen in Example 2, and demonstrate an additional variant that may be linked to hypercalcemia: PTH (44-77). As more disease states are scrutinized in more detail, it is expected that more unique PTH fragments will become evident and new disease-specific protein profiles will be established.

As demonstrated in the top-down analysis described above, a single, high-affinity polyclonal antibody was able to simultaneously extract numerous PTH variants and the selection of the epitope was directed by the desired assay target (ie intact and N-terminal variants). In the case presented here, our primary goal was to differentiate between intact PTH1-84 and N-terminal variant PTH7-84, while simultaneously identifying any additional N-terminal heterogeneity throughout the molecule. The results of these top-down experiments allowed the development of an initial standard profile for PTH. Clearly, this profile is not finite, and may be expanded to include additional variants found through literature search and/or complementary full-length studies. However, this standard profile provided an initial determination of target sequences for developing specific SRM assays.

This invention describes the development of a PTH MSIA that is capable of simultaneously monitoring full-length PTH and truncated variants with analytical metrics suitable for clinical research use. The primary object of these investigations was the design of assays able to capture data on two of these species—PTH1-84 (intact) and PTH7-84 (an N-terminal variant). Through judicious choice of antibody, the potentially confounding data from PTH1-34 (as it was not captured and did not enter into the analysis) was eliminated. Relative ion signals for these species confirmed that the assay was functional and created the basis for a standard PTH profile. This standard profile was expanded to include a peptide representative of a novel clinical variant PTH34-84, clipped at the N-terminus In its present form, 32 SRM transitions are analyzed in a multiplexed assay to monitor non-variant PTH sequence with >50% sequence coverage, as well as the two truncated variants. A systematic workflow allowed for the construction of an expandable variant map that presently contains an additional 8 molecular forms of PTH, including variants exhibiting conserved C-terminal truncations (i.e., truncations at PTH77). The construction of assays responsive to such possibilities is critical to achieving the most accurate qualitative and quantitative definitions of PTH and its related variants.

### REFERENCES

1. Aloia J F, Feuerman M, Yeh J K. Reference range for serum parathyroid hormone. Endocr Pract 2006;12:137-44.
2. Soldin S J, Brugnara C, Gunter K C, Et A. Pediatric Reference Ranges, 2nd ed, Vol. Washington D.C.: AACC Press, 1997:119.
3. Fraser W D. Hyperparathyroidism. Lancet 2009;374:145-58.
4. Brown R C, Aston J P, St John A, Woodhead J S. Comparison of poly- and monoclonal antibodies as labels in a two-site immunochemiluminometric assay for intact parathyroid hormone. J Immunol Meth 1988;109:139-44.
5. Endres D B, Villanueva R, Sharp C F, Jr., Singer F R. Immunochemiluminometric and immunoradiometric determinations of intact and total immunoreactive parathyrin: performance in the differential diagnosis of hypercalcemia and hypoparathyroidism. Clin Chem 1991;37:162-8.
6. Nussbaum S R, Zahradnik R J, Lavigne J R, Brennan G L, Nozawa-Ung K, Kim L Y, et al. Highly sensitive two-site immunoradiometric assay of parathyrin, and its clinical utility in evaluating patients with hypercalcemia. Clin Chem 1987;33:1364-7.
7. Boudou P, Ibrahim F, Cormier C, Chabas A, Sarfati E, Souberbielle J C. Third- or second-generation parathyroid hormone assays: a remaining debate in the diagnosis of primary hyperparathyroidism. J Clin Endocrinol and Metab 2005;90:6370-2.
8. Gao P, D'Amour P. Evolution of the parathyroid hormone (PTH) assay-importance of circulating PTH immunoheterogeneity and of its regulation. Clinical laboratory 2005;51:21-9.
9. Borges C R, Rehder D S, Jarvis J W, Schaab M R, Oran P E, Nelson R W. Thoughts on the Full-Length Characterization of Proteins in Human Population. Clin Chem 2010/In Press.
10. Langub M C, Monier-Faugere M C, Wang G, Williams J P, Koszewski N J, Malluche H H. Administration of PTH-(7-84) antagonizes the effects of PTH-(1-84) on bone in rats with moderate renal failure. Endocrinology 2003;144:1135-8.
11. D'Amour P. Circulating PTH molecular forms: what we know and what we don't. Kidney Int Suppl 2006:S29-33.
12. John M R, Goodman W G, Gao P, Cantor T L, Salusky I B, Juppner H. A novel immunoradiometric assay detects full-length human PTH but not amino-terminally truncated fragments: implications for PTH measurements in renal failure. J Clin Endocrinol Metab 1999;84:4287-90.
13. Salusky I B, Goodman W G. Adynamic renal osteodystrophy: is there a problem? J Am Soc Nephrol 2001;12:1978-85.
14. Winer K K, Yanovski J A, Cutler G B, Jr. Synthetic human parathyroid hormone 1-34 vs calcitriol and calcium in the treatment of hypoparathyroidism. JAMA 1996;276:631-6.
15. Neer R M, Arnaud C D, Zanchetta J R, Prince R, Gaich G A, Reginster J Y, et al.

Effect of parathyroid hormone (1-34) on fractures and bone mineral density in postmenopausal women with osteoporosis. New Eng J Med 2001;344:1434-41.
16. Miller P D, Bilezikian J P, Deal C, Harris S T, Ci R P. Clinical use of teriparatide in the real world: initial insights. Endocr Pract 2004;10:139-48.
17. Nelson R W. The use of bioreactive probes in protein characterization. Mass Spectrometry Reviews 1997;16:353-76.
18. Nelson R W, Krone J R, Bieber A L, Williams P. Mass spectrometric immunoassay. Anal Chem 1995;67:1153-8.
19. Kiernan U A, Nedelkov D, Tubbs K A, Niederkofler E E, Nelson R W. Selected expression profiling of full-length proteins and their variants in human plasma. Clin Proteomics J 2004;1:7-16.
20. Kiernan U A, Tubbs K A, Nedelkov D, Niederkofler E E, Nelson R W. Detection of novel truncated forms of human serum amyloid A protein in human plasma. FEBS Lett 2003;537:166-70.
21. Nedelkov D, Kiernan U A, Niederkofler E E, Tubbs K A, Nelson R W. Investigating diversity in human plasma proteins. Proc Natl Acad Sci U S A 2005;102:10852-7.
22. Nedelkov D, Phillips D A, Tubbs K A, Nelson R W. Investigation of human protein variants and their frequency in the general population. Mol Cell Proteomics 2007;6:1183-7.
23. Niederkofler E E, Kiernan U A, O'Rear J, Menon S, Saghir S, Protter A A, et al. Detection of Endogenous B-Type Natriuretic Peptide at Very Low Concentrations in Patients with Heart Failure. Circulation: Heart Failure 2008;1:258-64.
24. Zhang C X, Weber B V, Thammavong J, Grover T A, Wells D S. Identification of carboxyl-terminal peptide fragments of parathyroid hormone in human plasma at low-picomolar levels by mass spectrometry. Anal Chem 2006;78:1636-43.

25. Anderson N L, Anderson N G, Haines L R, Hardie D B, Olafson R W, Pearson T W. Mass spectrometric quantitation of peptides and proteins using stable isotope standards and capture by anti-peptide antibodies (SISCAPA). J Proteome Res 2004;3:235-44.
26. Berna M, Schmalz C, Duffin K, Mitchell P, Chambers M, Ackermann B.

Online immunoaffinity liquid chromatography/tandem mass spectrometry determination of a type II collagen peptide biomarker in rat urine: Investigation of the impact of collision-induced dissociation fluctuation on peptide quantitation. Anal Biochem 2006;356:235-43.
27. Oe T, Ackermann B L, Inoue K, Berna M J, Garner C O, Gelfanova V, Dean R A, Siemers E R, Holtzman D M, Farlow M R, Blair I A. Quantitative analysis of amyloid beta peptides in cerebrospinal fluid of Alzheimer's disease patients by immunoaffinity purification and stable isotope dilution liquid chromatography/negative electrospray ionization tandem mass spectrometry. Rapid Commun Mass Spectrom 2006;20:3723-35.
28. Ackermann B L, Berna M J. Coupling immunoaffinity techniques with MS for quantitative analysis of low-abundance protein biomarkers. Expert Rev Proteomics 2007;4:175-86.
29. Berna M, Ackermann B. Increased throughput for low-abundance protein biomarker verification by liquid chromatography/tandem mass spectrometry. Anal Chem 2009;81:3950-6.
30. Lopez M F, Kuppusamy R, Sarracino D A, Prakash A, Athanas M, Krastins B, et al. Discovery and targeted SRM assay development of first-trimester peptide biomarker candidates for Trisomy 21 in maternal blood. Mol Cell Proteomics In Press

We claim:

1. A method of detecting a parathyroid hormone(PTH)-related disease in a patient, comprising detecting the presence of one or more PTH variants in a biological sample from the patient, wherein the one or more PTH variants is selected from the group consisting of PTH 48-84 (SEQ ID NO: 1), 28-84 (SEQ ID NO: 6), 34-77 (SEQ ID NO: 7), 37-77 (SEQ ID NO: 8), 38-77 (SEQ ID NO:
9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO:
30), and 23-84 (SEQ ID NO: 22), and wherein the presence of one or more variants in the biological sample is indicative of a PTH-related disease in the patient.

2. The method of claim 1, wherein the one or more PTH variants is selected from the group consisting of PTH (48-84)(SEQ ID NO: 1), (28-84)(SEQ ID NO: 6), (34-77)(SEQ ID NO: 7), (37-77)(SEQ ID NO: 8), (38-77)(SEQ ID NO: 9), (34-79) (SEQ ID NO: 10), (41-84) (SEQ ID NO: 15), and 44-77 (SEQ ID NO: 30).

3. The method of claim 1, wherein the detecting the presence of one or more PTH variants in the biological sample comprises:
(a) capturing the one or more PTH variants using PTH affinity capture; and
(b) identifying specific PTH variants using a polypeptide separation assay.

4. The method of claim 1, wherein the detecting the presence of the one or more PTH variants in the biological sample comprises identifying the one or more PTH variants using binding molecules specific for each individual PTH variant to be detected.

5. The method of claim 1, wherein the method further comprises determining the quantity of the one or more PTH variants.

6. The method of claim 1, wherein the identity and/or quantity of the one or more PTH variants in the biological sample is indicative of treatment efficacy for the PTH-related disease in the patient.

7. The method of claim 5, wherein the quantity of the one or more PTH variants in the biological sample is indicative of disease progression of the PTH-related disease in the patient.

8. The method of claim 1, wherein the PTH-related disease is selected from the group consisting of primary hyperparathyroidism, secondary hyperparathyroidism, hypoparathyroidism, hypocalcemia, hypercalcemia, adynamic bone disease, vitamin D deficiency, renal osteodystrophy (chronic renal failure), diabetes, kidney disease, bone disease, osteoporosis, and parathyroid cancer.

9. The method of claim 1, wherein the PTH-related disease is renal osteodystrophy.

10. The method of claim 1 wherein the PTH variant is 44-77 (SEQ ID NO: 30).

11. The method of claim 10 wherein the PTH-related disease is hypercalcemia.

12. An isolated polypeptide selected from the group consisting of PTH 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO:
11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22).

13. An isolated binding molecule which selectively binds to a single isolated polypeptide according to claim 12.

14. The isolated binding molecule of claim 13, wherein the binding molecule is an antibody.

15. A pharmaceutical composition comprising:
(a) one or more substantially purified polypeptides selected from the group consisting of PTH variants 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO:
13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22); and
(b) a pharmaceutically acceptable carrier.

16. A method for treating a PTH-related disease, comprising administering to a patient in need thereof, a therapeutically effective amount to treat the PTH-related disease of one or more polypeptides selected from the group consisting of PTH fragments 48-84 (SEQ ID NO: 1), 28-84 (SEQ ID NO: 6), 34-77 (SEQ ID NO: 7), 37-77 (SEQ ID NO: 8), 38-77 (SEQ ID NO: 9), 34-79 (SEQ ID NO: 10), 38-76 (SEQ ID NO: 11), 38-79 (SEQ ID NO: 12), 44-84 (SEQ ID NO: 13), 38-80 (SEQ ID NO: 14), 41-84 (SEQ ID NO: 15), 30-78 (SEQ ID NO: 16), 27-75 (SEQ ID NO: 17), 26-77 (SEQ ID NO: 18), 19-70 (SEQ ID NO: 19), 9-75 (SEQ ID NO: 20), 4-82 (SEQ ID NO: 21), 44-77 (SEQ ID NO: 30), and 23-84 (SEQ ID NO: 22).

17. The method of claim 16 wherein the PTH-related disease is selected from the group consisting of primary hyperparathyroidism, secondary hyperparathyroidism, hypoparathyroidism, hypocalcemia, hypercalcemia, adynamic bone disease, vitamin D deficiency, renal osteodystrophy, chronic renal failure, diabetes, kidney disease, bone disease, osteoporosis, or parathyroid cancer.

18. A method for identifying polypeptide variants in a biological sample comprising:
(a) capturing the polypeptide variants from the biological sample using affinity capture; and
(b) identifying the polypeptide variants using a polypeptide separation assay.

19. The method of claim 18, wherein the method is used to identify PTH variants.

20. A method for identifying heterogeneity of PTH variants, in a sample, comprising:
(a) capturing the PTH variants using PTH affinity capture; and
(b) identifying one or more PTH variants using mass spectroscopy.
